(19) United States
(12) Patent Application Publication
(10) Pub. No.: US 2025/0302742 A1
(43) Pub. Date: Oct. 2, 2025

(54) A PHARMACEUTICAL FORMULATION FOR PRESSURISED METERED DOSE INHALER

(71) Applicant: CHIESI FARMACEUTICI S.P.A., Parma (IT)

(72) Inventors: Enrico ZAMBELLI, Parma (IT); Sauro BONELLI, Parma (IT); Angelo Benedetto MATTURRO, Parma (IT); Francesca USBERTI, Parma (IT); Alessandro CAVECCHI, Parma (IT)

(73) Assignee: CHIESI FARMACEUTICI S.P.A., Parma (IT)

(21) Appl. No.: 18/865,906

(22) PCT Filed: May 26, 2023
(86) PCT No.: PCT/EP2023/064262

§ 371 Date: Nov. 14, 2024

(30) Foreign Application Priority Data
May 27, 2022 (EP) 22175772.7

(51) Int. Cl. A61K 9/00; A61K 31/137; A61K 31/58; A61K 47/06; A61K 47/10; A61K 47/18; A61M 15/00

(52) U.S. Cl. CPC A61K 9/008; A61K 31/137; A61K 31/58; A61K 47/06; A61K 47/10; A61K 47/183; A61M 15/0001

(57) ABSTRACT

The present invention generally relates to pharmaceutical composition comprising a LABA agent, optionally in combination with other active ingredients, a chelating agent, a propellant and a co-solvent. The invention also provides a pharmaceutical composition for the treatment of respiratory diseases, such as asthma and COPD.

### FIELD OF THE INVENTION

The present invention generally relates to a pharmaceutical composition comprising a LABA agent, a chelating agent, a propellant and a co-solvent; the invention further relates to the use of such pharmaceutical compositions in the treatment and prevention of respiratory diseases.

### BACKGROUND OF THE INVENTION

Pressurized metered dose inhalers (pMDIs) are well known devices for administering pharmaceutical products to the respiratory tract by inhalation. A pMDI device typically presents a medical-containing canister (or a “can” as herein referred to), and an actuator housing having a mouthpiece. The can is usually crimped with a metered valve assembly. Depending on the active ingredients and on additional components such as excipients, acids and similar, a final pMDI formulation may be in the form of a solution or a suspension. As known in the art, solution is generally intended as substantially lacking precipitates or particles, while suspension typically refers to formulation having some undissolved material or precipitates. pMDI devices may use a propellant to expel droplets containing the pharmaceutical products to the respiratory tract as an aerosol.

Aerosol inhalation compositions suitable for a pMDI device comprising formoterol have been described in literatures.

WO 01/89480 describes a pharmaceutical composition comprising formoterol fumarate in a solution of HFA propellant and a co-solvent, containing an amount of HCl such that the solution has an apparent pH between 3 and 3.5.

EP 1480615B1 describes pharmaceutical formulation suitable for a pMDI administration comprising formoterol in a solution of a liquefied HFA propellant, ethanol, wherein the amount of residual water is less than 1500 ppm on the total weight of the formulation.

WO 2019/7236559 describes a pharmaceutical composition comprising formoterol fumarate, beclometasone dipropionate, an HFA propellant, a co-solvent, wherein the formulation is stabilized by the addition of an organic acid such as maleic acid.

WO 2011/076843 describes a stabilized pharmaceutical composition comprising formoterol, glycopyrronium bromide dissolved in HFA propellant and a co-solvent wherein the formulation contains an amount of 1M HCl comprised in the range 0.1-0.3 μg/μl.

WO 2015/101576 describes a pMDI device particularly suitable for the use with a formoterol, beclometasone dipropionate and glycopyrronium bromide solution, HCl, contained in a FEP coated can.

The chemical stability of the active pharmaceutical ingredients (APIs) contained in the pharmaceutical compositions is particularly desirable in order to obtain formulations suitable for the market, and to ensure the delivery of a constant dose of active ingredients per actuation.

Although the above-mentioned prior art provides effective formulations and devices technical arrangements, there is still the need to find an alternative aerosol formulation comprising a LABA agent particularly in combination with a corticosteroid, that is stable over an extended product lifetime.

We have surprisingly found that the inclusion of a chelating agent in a formulation comprising a LABA agent, optionally in combination with a corticosteroids, when the formulation is contained in a FEP coated canister substantially avoids the degradation of said active ingredients, thus maintaining the formulation stable over an extended period, and also exploiting an improvement in the stability profile of the formulation when suitable conditions are achieved.

Advantageously, said aerosol formulations comprising a chelating agent as herein described, when formulated in a propellant, in the presence of a co-solvent can be usable in a pMDI device, particularly for the treatment of respiratory diseases, such as asthma and/or COPD, with excellent aerosolizing performances.

### SUMMARY OF THE INVENTION

In one aspect, the present invention refers to a pharmaceutical composition comprising a

LABA agent, a co-solvent, a propellant and a chelating agent.

Particularly, the invention refers to such a formulation, also comprising a corticosteroid agent, and optionally a LAMA agent.

In a second aspect, the invention refers to a canister for a pMDI device, containing the pharmaceutical composition as above described, wherein said can is a FEP coated can.

In a further aspect, the invention refers to the use of said pharmaceutical composition comprising a LABA agent, a co-solvent, a propellant and a chelating agent for use as a medicament.

In an additional embodiment, the invention refers to a pMDI device containing the formulation of the invention.

In a further aspect, the invention further relates to the use of a pharmaceutical composition comprising a LABA agent, a co-solvent, a propellant and a chelating agent, for the treatment and/or prophylaxis of respiratory disorders, in particular asthma and COPD.

### DETAILED DESCRIPTION OF THE INVENTION

Unless otherwise defined, all technical and scientific terms used herein have the same meanings as commonly understood by the skilled in the art.

The “molar ratio” between formoterol or a salt thereof or a solvate of said salt and the acid is calculated considering the number of moles of formoterol or a salt thereof or a solvate of said salt within the formulation and number of moles of the selected acid in the formulation.

Unless otherwise indicated the term “LABA” or “LABA agent” includes in its meaning a long acting beta 2 agonist, as known in the art, such as formoterol fumarate, arformoterol, or fenoterol.

Unless otherwise provided, the term “formoterol fumarate” or “FF” refers to (R,R)-(±)formoterol fumarate or dihydrate thereof.

Unless otherwise indicated the term “EDTA” refers to ethylenediaminetetraacetic acid.

Unless otherwise indicated the term “EDTANa₄” or “tetrasodium EDTA” or “tetrasodium edetate” refers to the salt ethylenediaminetetraacetic acid with four sodium atoms.

Unless otherwise indicated the term “EDTANa₂” or “disodium EDTA” or “disodium edetate” refers a salt of ethylenediaminetetraacetic acid with two sodium atoms.

Unless otherwise indicated the term “EDTANa₂Ca” or “sodium calcium edetate” or “edetate calcium disodium” refers to a salt of ethylenediaminetetraacetic acid with two sodium and one calcium atoms.

Unless otherwise indicated the term “EDTACa” or “edetate monocalcium” refers to a salt of ethylenediaminetetraacetic acid with one calcium atom.

The term “% w/w” means the weight percentage of the component in respect to the total weight of the formulation.

The term “% w/v” means the weight percentage of the component in respect to the total volume of the formulation.

Regarding the term “apparent pH” as herein intended, it is noticed that the calculation of the pH is generally characteristic of aqueous liquid, e.g. where water is the dominant component. In relatively aprotic solvents (such as the propellants used in the present invention, e.g. an HFA or HFO system) protons are non-hydrated and their activity coefficients can differ from those in aqueous solution. Although the Nerst equation (describing potential of electrochemical cell as a function of concentrations of ions taking part in the reaction) with respect to electromagnetic field (EMF) applies and the pH-meter glass electrode system will generate a variable milli-volt output according to proton concentration and vehicle polarity, the pH meter reading represents the “apparent pH” according to the present invention. In this direction, the apparent pH according to the invention can be measured by technologies known in the art, as e.g. indicated in “Correlation between Apparent pH and Acid or Base Concentration in ASTM Medium” Orest Popovych, Analytical Chemistry 1964, 36,4,878-882; Analytical Standard Test Method (ASTM) D6423-19 “Standard Test Method for Determination of pH of Denatured Fuel Ethanol and Ethanol Fuel Blends”.

The term “chelating agent” refers to organic compounds capable of linking together metal ions to form complex ring-like structures called chelates, as e.g. indicated in Handbook of Toxicology of Chemical Warfare Agents, 2009.

As above mentioned, the present invention unexpectedly shows that the inclusion of a chelating agent in the formulation comprising a LABA agent, optionally in combination with a corticosteroid, stabilizes the thus obtained formulation, when contained in a FEP coated can.

In one embodiment, the formulation of the invention is characterized by comprising a chelating agent selected from the group consisting of EDTA, EDTANa₂, EDTANa₂Ca, EDTACa. More preferably the formulation comprises EDTANa₄.

In this respect, it has been surprisingly found that a formulation suitable for pMDI administration and comprising at least a LABA agent, and optionally a corticosteroid, is particularly stable when EDTANa₄ is used. From the data collected in the herein below experimental part, it is evident that the use of EDTANa₄ stabilizes the formulation in terms of % residual of active ingredient(s) when the formulation is contained in a FEP coated can. As shown in Tables 2, 3, 5 and 6 the addition of EDTANa₄ to a formulation comprising formoterol fumarate and BDP, contained in a FEP coated can, stabilizes the formulation in terms of % residue of the active ingredients, in particular formoterol fumarate. As it can be appreciated the chelating agent, is able to stabilize not only the formoterol fumarate, but also the other active ingredients contained in the formulation, such as the beclometasone dipropionate.

As further advantage, the formulation of the invention comprising the EDTANa₄ is characterized by the possibility to have a total amount of water higher than 1500 ppm.

Even further, we found that when the amount of water is more than 5000 ppm, the invention is particularly advantageous as indicated in the experimental part.

In a preferred embodiment, the water amount is comprised between 5000 and 13000 ppm, preferably between 6000 and 12000 ppm, more preferably between 10000 and 12000 ppm.

Generally, the amount of water is achieved by the presence of the aqueous solution of EDTANa₄ and/or by a proper addition of water.

The present invention brings several advantages to the prior art, such as the stability of the formulation over the time, good shelf life, good reproducibility of the final formulation, the maintenance of optimal chemical conditions within cans readily available in commerce, and a consistent efficacy of medication, particularly when formulated as a solution for a pMDI device.

In one embodiment the EDTANa₄ is added to the formulation as aqueous solution at concentration comprised between 1 and 5 mg/ml. Preferably the concentration is comprised between 2 and 4 mg/ml. More preferably the concentration is comprised between 2 and 3 mg/ml.

In one embodiment, the amount of EDTANa₄ contained in the pharmaceutical formulation is in a range from 0.00002 to 0.002% w/w. Preferably the amount of EDTANa₄ is in a range from 0.0001 to 0.0009% w/w; more preferably the amount of EDTANa₄ is in a range from 0.0001 to 0.0005% w/w; still more preferably the amount of EDTANa4 is in a range from 0.0002 to 0.0003 % w/w; even more preferably the amount of EDTANa₄ is 0.00025% w/w.

In one preferred embodiment, the formulation of the invention comprises a LABA agent, a chelating agent, preferably EDTANa₄, and a corticosteroid.

In one embodiment, the LABA agent of the formulation according to the invention, is selected from the group consisting of: fenoterol, formoterol fumarate, formoterol fumarate dihydrate, arformoterol, carmoterol (TA-2005), indacaterol, milveterol, bambuterol, clenbuterol, vilanterol, olodaterol, abediterol, terbutaline, salmeterol, diastereoisomeric mixtures, and a pharmaceutically acceptable salt thereof or hydrate thereof.

In one embodiment, the LABA is formoterol fumarate, preferably formoterol fumarate dihydrate.

In another embodiment, the formulation of the present invention comprises salbutamol, or (R)-salbutamol (levalbuterol) or a pharmaceutically acceptable salt thereof or hydrate thereof.

Preferably, the amount of LABA according to the present invention is comprised between 0.0005-0.04% w/w, more preferably between 0.001-0.03% w/w, even more preferably between 0.005-0.02% w/w.

In one embodiment, the corticosteroid component of the formulation according to the invention, is selected from the group consisting of: budesonide, beclometasone, e.g. as the mono or the dipropionate ester, flunisolide, fluticasone, e.g. as the propionate or furoate ester, ciclesonide, mometasone, e.g. as the furoate ester, mometasone desonide, rofleponide, hydrocortisone, prednisone, prednisolone, methyl prednisolone, naflocort, deflazacort, halopredone acetate, fluocinolone acetonide, fluocinonide, clocortolone, tipredane, prednicarbate, alclometasone dipropionate, halometasone, rimexolone, deprodone propionate, triamcinolone, betamethasone, fludrocortisone, desoxycorticosterone, rofleponide, etiprednol dicloacetate.

Beclometasone dipropionate (BDP) and budesonide are particularly preferred.

In a still preferred embodiment, the corticosteroid component is beclometasone dipropionate (BDP).

According to another embodiment of the present invention, the amount of the corticosteroid component, preferably BDP, is comprised between 0.01-0.7% w/w, more preferably between 0.05-0.5% w/w, even more preferably between 0.08-0.35% w/w.

In one embodiment, the present invention refers to a formulation, preferably a solution suitable for pMDI administration, comprising: a LABA agent, a corticosteroid and a chelating agent.

In one preferred embodiment, the present invention refers to a formulation, preferably a solution suitable for pMDI administration, comprising: a LABA agent, a corticosteroid and a chelating agent.

In a further preferred embodiment, the present invention refers to a formulation, preferably a solution suitable for pMDI administration, comprising: a LABA agent, a corticosteroid and EDTANa₄.

In a particularly preferred embodiment, the present invention refers to a formulation, preferably a solution suitable for pMDI administration, comprising formoterol fumarate, BDP and a chelating agent.

In a still preferred embodiment, the present invention refers to a formulation, preferably a solution, comprising formoterol fumarate, BDP, and EDTANa₄.

As above indicated, the formulation of the invention is particularly suitable for the administration as a pMDI solution. In this respect, the present formulation also comprises a propellant and preferably, a co-solvent, as herein below described.

The propellant of the formulation according to the invention is selected from hydrofluoroalkane (HFA) and hydrofluoroolefins (HFOs) and a mixture thereof.

In one embodiment, the hydrofluoroalkane propellant is selected from the group consisting of: HFA134a (1,1,1,2-tetrafluoroethane), HFA 227 (1,1,1,2,3,3,3-heptafluoropropane, HFA152a (1,1-Difluoroethane) and mixtures thereof.

In one embodiment, the HFO propellant of the formulation according to the invention is selected from the group consisting of: 1,3,3,3-tetrafluoropropene (HFO-1234ze) and 2,3,3,3-tetrafluoropropene (HFO-1234yf).

Preferably the propellant is an HFA propellant, more preferably HFA134a.

In an equally preferred embodiment, the propellant is HFA152a.

HFAs or HFOs may be present in the formulation in an amount in the range from 75 to 95% (w/w), preferably from 85 to 90% (w/w), based on the total weight of the formulation.

As above set forth, in one embodiment the formulation comprising a chelating agent according to the invention, may optionally further comprise additional components such as excipients, additives or low volatility components. The addition of said components may be suitably calibrated in order to modulate e.g. the chemical-physical properties of the formulation. In this respect, and also according to the above described preferred embodiments, the invention refers to a formulation as above described in detail, also comprising an HFA or HFO propellant, a co-solvent and optionally a low volatile component.

Preferably, said co-solvent is a polar compound able to increase the solubility of the components within the formulation. Preferred co-solvents are aliphatic alcohols having from 1 to 4 carbon atoms, such as methanol, ethanol, propanol, isopropanol and the like, preferably ethanol, more preferably anhydrous ethanol.

When present, said co-solvent is used in an amount comprised from 5% w/w and 20% w/w, more preferably from 10% and 15% w/w, based on the total weight of the formulation.

When present, the low volatility component is a compound characterized in having a vapor pressure at 25° C. lower than 0.1 kPa, preferably lower than 0.05 kPa. Preferred low volatility components are selected from the group consisting of glycols, propylene glycol, polyethylene glycol, glycerol or esters thereof, ascorbyl palmitate and isopropyl myristate, wherein isopropyl myristate and glycerol are particularly preferred.

In a preferred embodiment, the present invention refers to a formulation, preferably a solution suitable for pMDI administration, comprising, consisting of or consisting essentially of: a LABA agent and a corticosteroid, a chelating agent, a propellant and an aliphatic alcohol having from 1 to 4 carbon atoms, preferably ethanol, more preferably anhydrous ethanol.

In a further preferred embodiment, the present invention refers to a formulation, preferably a solution suitable for pMDI administration, comprising, consisting of or consisting essentially of: a LABA agent and a corticosteroid, a chelating agent, an HFA propellant and an aliphatic alcohol having from 1 to 4 carbon atoms, preferably ethanol.

In a still preferred embodiment, the present invention refers to a formulation, preferably a solution suitable for pMDI administration, comprising, consisting of or consisting essentially of: formoterol fumarate, BDP, EDTANa₄, an HFA propellant, preferably HFA 134a or HFA 152a and ethanol, more preferably anhydrous ethanol.

In one embodiment, the present invention refers to a formulation, preferably a solution suitable for pMDI administration, comprising, consisting of or consisting essentially of: formoterol fumarate, BDP, an amount of EDTANa₄ in a range from 0.00002 to 0.002% w/w, an HFA propellant selected from HFA 134a and HFA 152a, and ethanol, preferably anhydrous ethanol.

In one preferred embodiment, the present invention refers to a formulation, preferably a solution suitable for pMDI administration, comprising, consisting of or consisting essentially of: formoterol fumarate, BDP, an amount of EDTANa₄ in a range from 0.0001 to 0.0009% w/w, an HFA propellant selected from HFA 134a and HFA 152a, and ethanol, preferably anhydrous ethanol.

In a further preferred embodiment, the present invention refers to a formulation, preferably a solution suitable for pMDI administration, comprising, consisting of or consisting essentially of: formoterol fumarate, BDP, an amount of EDTANa₄ in a range from 0.0001 to 0.0005% w/w, preferably from 0.0002 to 0.0003% w/w, an HFA propellant selected from HFA 134a and HFA 152a, and ethanol, preferably anhydrous ethanol.

In a particularly preferred embodiment, the present invention refers to a formulation, preferably a solution suitable for pMDI administration, comprising, consisting of or consisting essentially of: formoterol fumarate, BDP, an amount of EDTANa₄ of 0.000025% w/w, HFA 134a and ethanol, preferably anhydrous ethanol.

In an equal preferred embodiment, the present invention refers to a formulation, preferably a solution suitable for pMDI administration, comprising, consisting of or consisting essentially of: formoterol fumarate, BDP, an amount of EDTANa₄ of 0.000025% w/w, HFA 152a and ethanol, preferably anhydrous ethanol.

According to the invention, the present formulation can be a solution, a suspension or a system comprising solution and suspension.

In a preferred embodiment, the formulation of the invention is a solution. Preferably one or more (more preferably all) of the pharmaceutically active components of the formulation, e.g. the LABA and/or corticosteroid are completely and homogenously dissolved in the propellant and co-solvent.

As far as the can or canister is concerned, part or all of the canister of the pMDI device suitable to contain the formulation of the invention, may be made of a metal, e.g. aluminum, or metal alloys, stainless steel or anodized aluminum, fluorine passivated aluminum and the like. Alternatively, the canister may be a plastic can or a plastic-coated glass bottle.

The metal canisters may have part or all of the internal surfaces lined with an inert organic coating.

The coating is typically applied to the internal surface of the can, thus providing an internal layer acting as interface between the internal surface of the can, and the formulation therein contained.

In this regards, a suitable coated can of the invention may have part or all of its internal surfaces coated with an inert organic or inorganic coating preferably comprising: an epoxy-phenol resin, a perfluorinated polymer, a perfluoroalkoxyalkane polymer, a perfluoroalkoxyalkylene polymer (PFA), a perfluoroalkylene polymer, poly-tetrafluoroethylene polymer (PTFE or Teflon), fluorinated-ethylene-propylene polymer (FEP), polyether sulfone polymer (PES), a fluorinated-ethylene-propylene polyether sulfone polymer (FEP-PES), a polyamide, polyimide, polyamideimide, polyphenylene sulfide, plasma, mixtures or combinations thereof.

In a preferred embodiment, the invention refers to the above described formulation, contained in a FEP coated cans.

In another embodiment, the present invention refers to a formulation, preferably a solution suitable for pMDI administration, comprising, consisting of or consisting essentially of: formoterol fumarate, BDP, EDTANa₄, an HFA propellant, preferably HFA 134a or HFA 152a and ethanol, more preferably anhydrous ethanol, contained in a FEP coated can.

In one embodiment, the invention refers to a pMDI device comprising a FEP coated can filled with the formulation, preferably a solution comprising, consisting of or consisting essentially of: formoterol fumarate, BDP, EDTANa₄, an HFA propellant, preferably HFA 134a or HFA 152a and ethanol, more preferably anhydrous ethanol.

The canister of a pMDI device is typically crimped with a metering valve for delivering a therapeutically effective dose of the active ingredients.

The metering valve assembly comprises at least one rubber gasket seal made of a proper elastomeric material selected from: low-density polyethylene, butyl or halo butyl rubbers such as chlorobutyl or bromubutyl rubbers (optionally halogenated copolymers of isobutylene with isoprene), butadiene-acrylonitrile, neoprene, EPDM (a polymer of ethylenepropylenediene monomer), TPE (thermoplastic elastomer), cycloolefin copolymer (COC) or combination thereof.

The metering valve according to the invention is typically capable of delivering a volume in the range from 25 to 150 μl, preferably in the range from 50 to 100 μl, and more preferably from 50 μl to 70 μl per actuation; the most preferred are 50, 63 and 100 μl per actuation. Suitable valves for the present invention are commercially available.

According to a further aspect of the invention there is provided a method of filling an aerosol inhaler with a pharmaceutical composition of the invention. Conventional bulk manufacturing methods and machinery well known to those skilled in the art of pharmaceutical aerosol manufacture may be employed for the preparation of large-scale batches for the commercial production of filled canisters.

As a general example said methodology may comprise the steps of:
a) preparing a solution comprising: formoterol fumarate, BDP and ethanol;
b) adding the amount of EDTANa₄ (as aqueous solution) to the ethanolic solution and mix the bulk solution;
c) filling the canister with said solution;
d) crimping with a valve and gassing with HFA propellant.

The packaged formulations of the invention are stable for extended periods of time when stored under normal conditions of temperature and humidity. Stability is assessed by measuring content of residual active ingredient(s).

In a further aspect, the invention refers to the above described formulation for use as a medicament. Thus, the invention refers to the use of the formulation as herein described for the preparation of a medicament.

Preferably, the formulation of the invention is for prophylactic purposes or for symptomatic relief of a wide range of respiratory disorders, such as asthma of all types and chronic obstructive pulmonary disease (COPD).

In one preferred embodiment, the invention refers to the formulation as herein described, for the treatment and/or prophylaxis of respiratory disorders, preferably for the treatment and/or prophylaxis of asthma or COPD.

Other respiratory disorders for which use of the pharmaceutical compositions of the invention may be beneficial are those characterized by obstruction of the peripheral airways as a result of inflammation and presence of mucus, such as chronic obstructive bronchiolitis, chronic bronchitis, emphysema, acute lung injury (ALI), cystic fibrosis, rhinitis, and adult or acute respiratory distress syndrome (ARDS).

As it is will be recognized, all the herein described embodiments are to be intended as included in the scope of the present invention, also in any possible combination with all the other preferred embodiments, as herein above and below set forth.

The invention will be now described by the following not limiting examples.

### EXPERIMENTAL PART

### Example 1

A study was performed to investigate the chemical stability of formulation intended for pMDI administration comprising formoterol fumarate dihydrate (FF) and beclometasone dipropionate (BDP). Said formulation is a solution, contained in a FEP coated can crimped with a metering valve having a 50 μl metering volume.

An amount of EDTANa₄ was added to the formulation, thus providing Formulation 1, as reported in Table 1.

TABLE 1

| COMPONENT | Formulation 1 (% w/w) |
|---|---|
| FF | 0.010 |
| BDP | 0.172 |
| EDTANa₄ | 0.00025 |
| Ethanol anhydrous | 12.00 |
| H₂O | 1 |
| HFA 134a | 86.818 |

The Formulation 1 was put in stability chambers in inverted position at 40° C., 75% R.H. for 1 month, the API assay and relevant degradation products were measured at T1 (1 month).

The formulation was also tested at different stability conditions, at 25° C., 60% R.H. for 6 months, the API assay and relevant degradation products were measured at T3 (3 months).

APIs residue % are reported in Table 2 and 3.

TABLE 2

T1 40° 75%

| | | FF | BDP | Apparent | Water |
|---|---|---|---|---|---|
| Formulation | Can | % | % | pH | (ppm) |
| 1 | FEP | 94.7 | 97.8 | 6.3 | 11354 |

TABLE 3

T3 25°/60%

| | | FF | BDP | Apparent | Water |
|---|---|---|---|---|---|
| Formulation | Can | % | % | pH | (ppm) |
| 1 | FEP | 97.6 | 97.8 | 6.4 | 11399 |

As it can be observed by Tables 2 and 3 when the EDTANa₄ is added according to Formulation 1, the chemical stability of formoterol (FF) and beclometasone dipropionate (BDP) is achieved even in the presence of a residual water content greater than 1500 ppm. Of note, the % FF residue can reach values even higher than 90%.

### Example 2

A second study was performed to investigate the chemical stability of formulation intended for pMDI administration comprising formoterol fumarate dihydrate (FF) and beclometasone dipropionate (BDP) in an HFA152a propellant. Said formulation is a solution, contained in a FEP coated can crimped with a metering valve having a 63 μl metering volume.

An amount of EDTANa₄ was added to the formulation, thus providing Formulation 2, as reported in Table 4.

TABLE 4

| COMPONENT | Formulation 2 (% w/w) |
|---|---|
| FF | 0.0107 |
| BDP | 0.1788 |
| EDTANa₄ | 0.0003 |
| Ethanol anhydrous | 10 |
| H₂O | 0.5 |
| HFA 152a | 89.2 |

The Formulation 2 was put in stability chambers in inverted position at 40° C., 75% R.H. for 1 month, the API assay and relevant degradation products were measured at T1 (1 month).

The formulation was also tested at different stability conditions, at 25° C., 60% R.H. for 3 months, the API assay and relevant degradation products were measured at T3 (3 months).

APIs residue % are reported in Table 5 and 6.

TABLE 5

T1 40° 75%

| | | FF | BDP | Apparent | Water |
|---|---|---|---|---|---|
| Formulation | Can | % | % | pH | (ppm) |
| 2 | FEP | 96.8 | 100.0 | 6.32 | 6170 |

TABLE 6

T3 25°/60%

| | | FF | BDP | Apparent | Water |
|---|---|---|---|---|---|
| Formulation | Can | % | % | pH | (ppm) |
| 2 | FEP | 98.5 | 100.0 | 6.33 | 6219 |

As it can be observed by Tables 5 and 6 when the EDTANa₄ is added according to Formulation 2, the chemical stability of formoterol (FF) and beclometasone dipropionate (BDP) is achieved even in the presence of a residual water content greater than 1500 ppm. Of note, the % FF residue can reach values even higher than 90%.

1. A pharmaceutical composition comprising a LABA agent, a co-solvent, a propellant and a chelating agent.

2. The pharmaceutical composition according to claim 1 wherein the LABA agent is selected from the group consisting of fenoterol, formoterol fumarate, formoterol fumarate dihydrate, arformoterol, carmoterol (TA-2005), indacaterol, milveterol, bambuterol, clenbuterol, vilanterol, olodaterol, abediterol, terbutaline, salmeterol, diastereoisomeric mixtures, and a pharmaceutically acceptable salt thereof or hydrate thereof.

3. The pharmaceutical composition according to claim 1, wherein the LABA agent is formoterol fumarate dihydrate.

4. The pharmaceutical composition according to claim 3, wherein the LABA agent is formoterol fumarate.

5. The pharmaceutical composition according to claim 1, wherein the chelating agent is selected from the group consisting of EDTA, EDTANa₂, EDTANa₂Ca, EDTACa, and EDTANa₄.

6. The pharmaceutical composition according to claim 1, wherein the amount of EDTANa₄ is from 0.00002 to 0.002% w/w.

7. The pharmaceutical composition according to claim 6, wherein the amount of EDTANa₄ is from 0.0001 to 0.0009% w/w.

8. The pharmaceutical composition according to claim 7, wherein the amount of EDTANa₄ is from 0.0001 to 0.0005% w/w.

9. The pharmaceutical composition according to claim 8, wherein the amount of EDTANa₄ is from 0.0002 to 0.0003% w/w.

10. The pharmaceutical composition according to claim 9, wherein the amount of EDTANa₄ is 0.00025% w/w.

11. The pharmaceutical composition according to claim 1, further comprising a corticosteroid selected form the group consisting of budesonide, beclometasone (BDP), as the mono or the dipropionate ester, flunisolide, fluticasone, as the propionate or furoate ester, ciclesonide, mometasone, as the furoate ester, mometasone desonide, rofleponide, hydrocortisone, prednisone, prednisolone, methyl prednisolone, naflocort, deflazacort, halopredone acetate, fluocinolone acetonide, fluocinonide, clocortolone, tipredane, prednicarbate, alclometasone dipropionate, halometasone, rimexolone, deprodone propionate, triamcinolone, betamethasone, fludrocortisone, desoxycorticosterone, rofleponide, etiprednol dicloacetate.

12. The pharmaceutical composition according to claim 11, wherein the corticosteroid is budesonide or beclometasone dipropionate (BDP).

13. The pharmaceutical composition according to claim 12, wherein the corticosteroid is beclometasone dipropionate (BDP).

14. The pharmaceutical composition according to claim 1, wherein the co-solvent is an aliphatic alcohol having from 1 to 4 carbon atoms.

15. The pharmaceutical composition according to claim 14, wherein the co-solvent is ethanol.

16. The pharmaceutical composition according to claim 1, wherein the propellant is selected from the group consisting of a hydrofluoroalkane (HFA), a hydrofluoroolefin (HFO), and a mixture thereof.

17. The pharmaceutical composition according to claim 16, wherein the propellant is selected from the group consisting of HFA134a, HFA152a, and mixtures thereof.

18. The pharmaceutical composition according to claim 17 wherein the propellant is HFA134a.

19. The pharmaceutical composition according to claim 17, wherein the propellant is HFA152a.

20. The pharmaceutical composition according to claim1, wherein the composition is a solution.

21. The pharmaceutical composition according to claim 1, wherein the LABA agent is formoterol fumarate dihydrate, the corticosteroid is beclometasone dipropionate (BDP), the propellant is HFA134a, the inorganic acid is HCl, the chelating agent is EDTANa₂, the co-solvent is ethanol, and the composition is a solution.

22. The pharmaceutical composition according to claim 1, wherein the LABA agent is formoterol fumarate dihydrate, the corticosteroid is budesonide or beclometasone dipropionate (BDP), the propellant is HFA152a, the inorganic acid is HCl, the chelating agent is EDTANa₂, the co-solvent is ethanol and the composition is a solution.

23. The pharmaceutical composition according to claim 1, wherein the total amount of water is from 5000 to 13000 ppm.

24. A canister comprising the pharmaceutical composition according to claim 1, wherein the canister is internally coated by a coating comprising at least a compound selected from the group consisting of an epoxy-phenol resin, a perfluorinated polymer, a perfluoroalkoxyalkane polymer, a perfluoroalkoxyalkylene polymer, a perfluoroalkylene polymer, poly-tetrafluoroethylene polymer (Teflon), fluorinated-ethylene-propylene polymer (FEP), polyether sulfone polymer (PES), a fluorinated-ethylene-propylene polyether sulfone polymer (FEP-PES), a polyamide, polyimide, polyamideimide, polyphenylene sulfide, plasma, and mixtures thereof.

25. The canister comprising the pharmaceutical composition according to claim 24, wherein the canister is a FEP coated can.

26. A canister for a pMDI device, containing a pharmaceutical composition according to claim 1.

27. A canister for a pMDI device according to claim 26 wherein the canister is a FEP coated can.

28. A pMDI device comprising a FEP coated can, containing a pharmaceutical composition according to claim 1.

29. (canceled)

30. A method for the treatment and/or prophylaxis of a respiratory disorder, comprising administering an effective amount of a pharmaceutical composition according to claim 1 to a subject in need thereof.

31. A method according to claim 30, wherein said respiratory disorder is asthma or COPD.
